(19) United States
(12) Patent Application Publication
(10) Pub. No.: US 2025/0121177 A1
(43) Pub. Date: Apr. 17, 2025

(54) INTRACARDIAC BLOOD PUMP WITH CAPACITIVE SENSING LOCATION DETECTION

(71) Applicant: Abiomed, Inc., Danvers, MA (US)

(72) Inventor: Dustin West, Danvers, MA (US)

(73) Assignee: Abiomed, Inc., Danvers, MA (US)

(21) Appl. No.: 18/917,215

(22) Filed: Oct. 16, 2024

Related U.S. Application Data
(60) Provisional application No. 63/590,902, filed on Oct. 17, 2023.

(51) Int. Cl. A61M 60/816; A61M 60/122

(52) U.S. Cl. CPC A61M 60/816; A61M 60/122; A61M 2205/04; A61M 2205/3317; A61M 2205/3327; A61M 2205/502; A61M 2210/125

(57) ABSTRACT

Methods and apparatus for determining a position of an intracardiac blood pump in a patient are described. The method includes receiving from at least one capacitive sensor associated with a cannula of the intracardiac blood pump, at least one capacitive signal, determining a position of the intracardiac blood pump in the patient based, at least in part, on the at least one capacitive signal, and providing an indication of the position of the intracardiac blood pump on a user interface of the circulatory support device.

### RELATED APPLICATIONS

This application claims the benefit under 35 U.S.C. § 119 (c) to U.S. Provisional Patent Application No. 63/590,902, filed Oct. 17, 2023 titled, “INTRACARDIAC BLOOD PUMP WITH CAPACITIVE SENSING LOCATION DETECTION,” the entire contents of which is incorporated by reference herein.

### FIELD OF THE INVENTION

This disclosure relates to an intracardiac blood pump with capacitive sensing location detection.

### BACKGROUND

Cardiovascular diseases are a leading cause of morbidity, mortality, and burden on global healthcare. A variety of treatment modalities have been developed for heart health, ranging from pharmaceuticals to mechanical devices and transplantation. Temporary cardiac support devices, such as heart pump systems (also referred to as “intracardiac blood pumps”), provide hemodynamic support and facilitate heart recovery. Intracardiac blood pumps have traditionally been used to temporarily assist the pumping function of a patient's heart during emergent cardiac procedures, such as a stent placement, performed after the patient suffers a heart attack, cardiac arrest, and/or cardiogenic shock. Intracardiac blood pumps also may be used to take the load off of a patient's heart to allow the heart to recover from such a cardiac procedure or from a heart attack, cardiac arrest, cardiogenic shock, or heart damage (e.g., caused by a viral infection). In that regard, an intracardiac blood pump can be introduced into the heart either surgically or percutaneously and used to deliver blood from one location in the heart or circulatory system to another location in the heart or circulatory system. For example, when deployed in the left heart, an intracardiac blood pump can pump blood from the left ventricle of the heart into the aorta. Likewise, when deployed in the right heart, an intracardiac blood pump can pump blood from the inferior vena cava into the pulmonary artery. Intracardiac pumps can be powered by a motor located outside of the patient's body via an elongate drive shaft (or drive cable) or by an onboard motor located inside the patient's body. Examples of such devices include the Impella® family of devices (Abiomed, Inc., Danvers, MA).

### SUMMARY

Described herein are systems and methods for determining a position of an intracardiac blood pump when placed within a patient. One or more capacitive sensors may be arranged proximate to the cannula of the blood pump, and one or more capacitive sensor signals sensed by the capacitive sensor(s) may be used to infer the position of the intracardiac blood pump in the patient's anatomy.

In some embodiments, a circulatory support device is provided. The circulatory support device includes an intracardiac blood pump comprising an inlet, an outlet, and a cannula arranged between the inlet and the outlet, at least one capacitive sensor associated with the cannula, and at least one controller. The at least one controller is configured to determine a position of the intracardiac blood pump in a patient based, at least in part, on at least one capacitive signal sensed by the at least one capacitive sensor, and provide an indication of the position of the intracardiac blood pump on a user interface of the circulatory support device.

In one aspect, the circulatory support device further includes a capacitive material surrounding the cannula, and a signal generator configured to impart an electromagnetic field surrounding the capacitive material. In another aspect, the at least one capacitive signal corresponds to at least one interference signal sensed when an object enters the electromagnetic field. In another aspect, the circulatory support device further includes an analog-to-digital converter configured to transform the at least one interference signal to a digital signal, and the at least one controller is further configured to determine the position of the intracardiac blood pump based, at least in part, on the digital signal. In another aspect, the at least one controller is further configured to determine the position of the intracardiac blood pump based, at least in part, on a comparison of the at least one capacitive signal with plurality of stored signal representations.

In another aspect, the circulatory support device further includes a shielding material arranged adjacent to the at least one capacitive sensor. In another aspect, the at least one capacitive sensor comprises a plurality of capacitive sensors, each of which is configured to sense a respective capacitive signal, and the at least one controller is further configured to determine a position of the intracardiac blood pump in a patient based, at least in part, on the plurality of capacitive signals. In another aspect, the at least one controller is further configured to determine a position of the intracardiac blood pump in a patient based, at least in part, on the plurality of capacitive signals by comparing each of the plurality of capacitive signals to a stored signal representation, and determining the position of the intracardiac blood pump based, at least in part, on the comparisons. In another aspect, the at least one controller is further configured to determine a position of the intracardiac blood pump in a patient based, at least in part, on the plurality of capacitive signals by combining each of the plurality of capacitive signals to generate a combined signal, and determining the position of the intracardiac blood pump based, at least in part, on a comparison of the combined signal with plurality of stored signal representations. In another aspect, the at least one controller is further configured to determine based, at least in part, on the at least one capacitive signal, an anatomy type of a tissue proximal to the cannula. In another aspect, the at least one controller is further configured to determine a position of the intracardiac blood pump in a patient based, at least in part, on the determined anatomy type.

In some embodiments, a method of determining a position of an intracardiac blood pump in a patient is provided. The method includes receiving from at least one capacitive sensor associated with a cannula of the intracardiac blood pump, at least one capacitive signal, determining a position of the intracardiac blood pump in the patient based, at least in part, on the at least one capacitive signal, and providing an indication of the position of the intracardiac blood pump on a user interface.

In one aspect, the method further includes generating, using a signal generator, an electromagnetic field proximate to the cannula, and wherein the at least one capacitive signal corresponds to at least one interference signal sensed when an object enters the electromagnetic field. In another aspect, the method further includes transforming the at least one interference signal to a digital signal, and determining the position of the intracardiac blood pump comprises determining the position of the intracardiac blood pump based, at least in part, on the digital signal. In another aspect, determining the position of the intracardiac blood pump comprises determining the position of the intracardiac blood pump based, at least in part, on a comparison of the at least one capacitive signal with plurality of stored signal representations.

In another aspect, receiving at least one capacitive signal comprises receiving a plurality of capacitive signals from a plurality of capacitive sensors associated with the cannula of the intracardiac blood pump, and determining a position of the intracardiac blood pump comprises determining the position in a patient based, at least in part, on the plurality of capacitive signals. In another aspect, determining a position of the intracardiac blood pump comprises determining the position of the intracardiac blood pump by comparing each of the plurality of capacitive signals to a stored signal representation, and determining the position of the intracardiac blood pump based, at least in part, on the comparisons. In another aspect, determining a position of the intracardiac blood pump comprises determining the position of the intracardiac blood pump by combining each of the plurality of capacitive signals to generate a combined signal, and determining the position of the intracardiac blood pump based, at least in part, on a comparison of the combined signal with plurality of stored signal representations. In another aspect, the method further includes determining based, at least in part, on the at least one capacitive signal, an anatomy type of a tissue proximal to the cannula. In another aspect, determining a position of the intracardiac blood pump comprises determining the position of the intracardiac blood pump based, at least in part, on the determined anatomy type.

### BRIEF DESCRIPTION OF DRAWINGS

FIG. 1A illustrates a pump system in accordance with some embodiments of the present disclosure.

FIG. 1B is a cross-sectional view of a portion of the pump system of FIG. 1A.

FIG. 2 illustrates a pump system in accordance with some embodiments of the present disclosure.

FIG. 3 illustrates a pump system in accordance with some embodiments of the present disclosure.

FIG. 4 schematically illustrates a capacitive sensing system that may be used to determine a position of an intracardiac blood pump in accordance with some embodiments of the present disclosure.

FIG. 5 illustrates a flowchart of a process for determining a position of an intracardiac blood pump in accordance with some embodiments of the present disclosure.

### DETAILED DESCRIPTION

Determining the position of an intracardiac blood pump is important during insertion of the pump to ensure that the pump is placed in a desired position (e.g., across the aortic valve, across the pulmonary valve) prior to operation. During operation, the pump may require repositioning to a desired position due to patient movements (e.g., patient movements that occur when the patient is transferred from the operating room to the intensive care unit (ICU), etc.) or due to other factors. Determining the position of the pump is typically accomplished using imaging to visualize the placement of the pump relative to cardiac structures in a patient. However, the inventor has recognized that imaging may not always be readily available (e.g., when the patient is in the ICU) and that other techniques that do not require visualization of the blood pump, but instead rely on indirect determination of the pump position, may also be advantageous. To this end, some embodiments of the present disclosure relate to techniques for determining the position of an intracardiac blood pump using one or more capacitive sensors associated with the cannula of the pump.

To provide an overall understanding of the systems, methods, and devices described herein, certain illustrative examples will be described. Although various examples may describe specific medical procedures and/or uses of intracardiac blood pumps, it will be understood that the technology described herein may be employed in any suitable context.

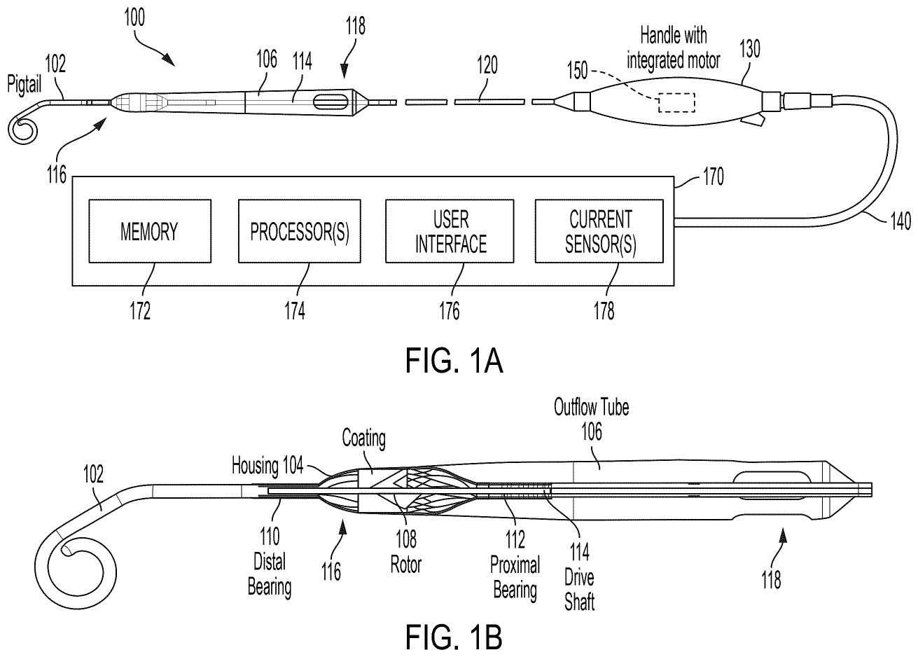

A pump system 100 (also referred to herein simply as pump 100) for use with some embodiments of the present disclosure is shown in FIGS. 1A and 1B. As shown, pump system 100 may be coupled to a control unit 170. Pump 100 may include a distal atraumatic tip 102, a pump housing 104 surrounding a rotor 108, an outflow tube 106, distal bearing 110, proximal bearing 112, inlet 116, outlet 118, catheter 120, handle 130, cable 140, and motor 150. As will be appreciated, although shown with an atraumatic tip, in some embodiments, the pump may not include such a tip. Pump housing 104 may be configured as a frame structure formed by a mesh with openings which may, at least in part, be covered by an elastic material. As will be appreciated, although shown as a frame structure, in some embodiments, the pump housing may be solid. A proximal portion of pump housing 104 may extend into and be mounted in the hollow interior of outflow tube 106, and a distal portion of pump housing 104 may extend distally beyond the distal end of outflow tube 106. The exposed openings in the pump housing 104 extending distally beyond outflow tube 106 may form the inlet 116 of pump 100. The proximal end of outflow tube 106 may include a plurality of openings that form the outlet 118 of pump 100. Rotor 108 may be rotationally mounted between distal bearing 110 and proximal bearing 112, and may be coupled to a distal end of drive shaft 114. Drive shaft 114 may be flexible and may extend through catheter 120, through the hollow interior of outflow tube 106, into handle 130 and is coupled to motor 150, which is housed in handle 130. In some embodiments, the motor 150 may be located inside of the patient. The proximal end of handle 130 may be coupled via cable 140 to control unit 170. A fluid may be circulated through the catheter 120 proximate to the drive shaft 114 and in the space surrounding the distal bearing 110 and proximal bearing 112 to lubricate those components and reduce friction during operation of the pump 100.

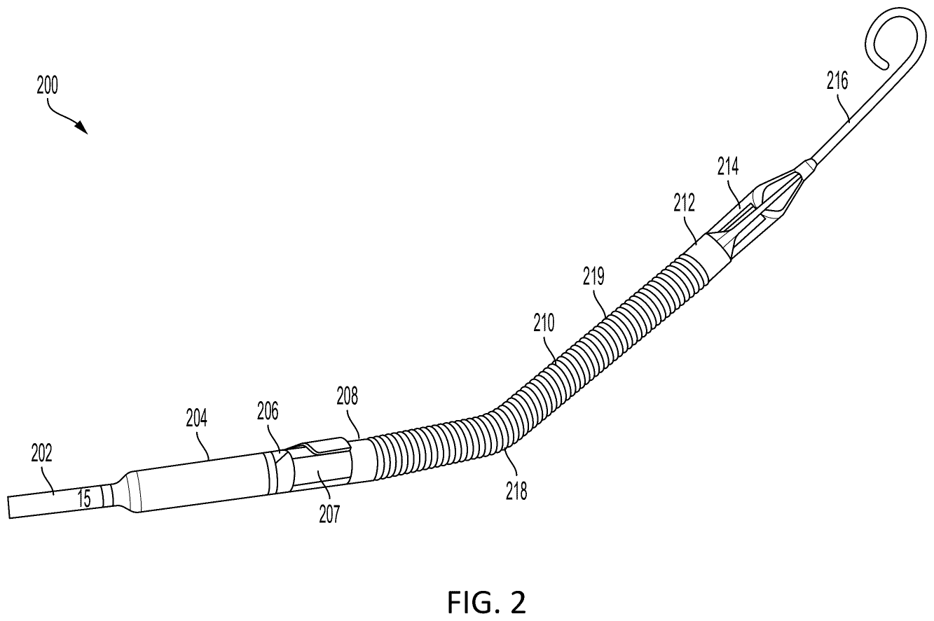

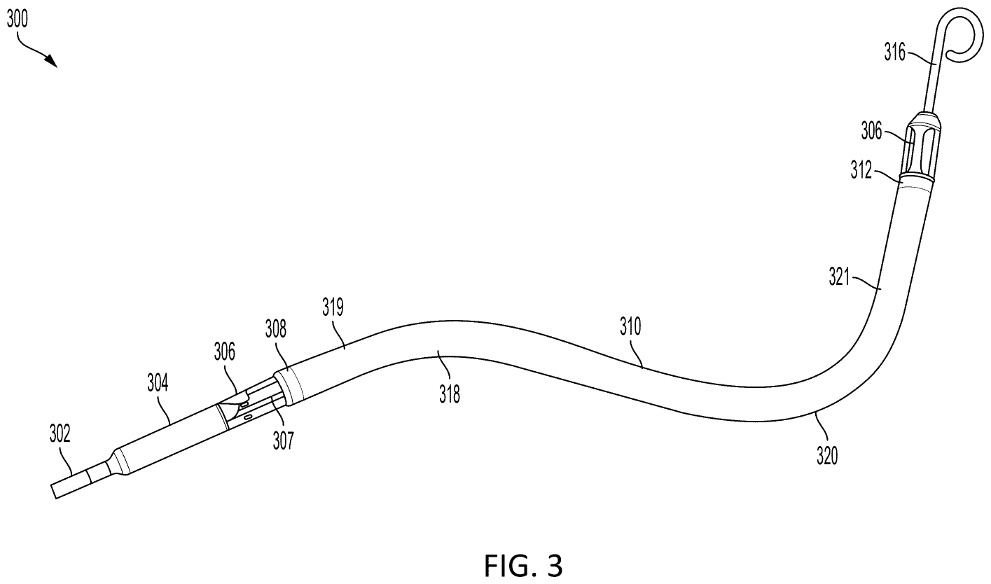

Control unit 170 may include one or more memory 172, one or more processors 174, a user interface 176, and one or more sensors, such as current sensors 178. Processor(s) 174 may comprise one or more microcontrollers, one or more microprocessors, one or more application specific integrated circuits (ASICs), one or more digital signal processors, program memory, or other computing components. Processor(s) 174 may be communicatively coupled to the other components (e.g., memory 172, user interface 176, current sensor(s) 178) of control unit 170 and may be configured to control one or more operations of pump 100. As will be appreciated, although control unit 170 is shown connected to pump 100, control unit may be connected to pump 200 or pump 300 described in connection with FIGS. 2 and 3. As a non-limiting example, control unit 170 may be implemented as an Automated Impella Controller™ from ABIOMED, Inc., Danvers, MA. In some aspects, memory 172 is included as a portion of processor(s) 174 rather than being provided as a separate component.

During operation, processor(s) 174 may be configured to control the electrical power delivered to motor 150 (e.g., by controlling a power supply (not shown)) by a power supply line (not shown) in cable 140, thereby controlling the speed of the motor 150. Current sensor(s) 178 may be configured to sense motor current associated with an operating state of the motor 150, and processor(s) 174 may be configured to receive the output of current sensor(s) 408 as a motor current signal. Processor(s) 174 may further be configured to determine a flow through the pump 100 based, at least in part, on the motor current signal and the motor speed, as described in more detail herein. Current sensor(s) 178 may be included in control unit 170 or may be located along any portion of the power supply line in cable 140. Additionally or alternatively, current sensor(s) 178 may be included in motor 150 and processor(s) 174 may be configured to receive the motor current signal via a data line (not shown) in cable 140 coupled to processor(s) 174 and motor 150.

Memory 172 may be configured to store computer-readable instructions and other information for various functions of the components of control unit 170. In one aspect, memory 172 includes volatile and/or non-volatile memory, such as, an electrically erasable programmable read-only memory (EEPROM).

User interface 176 may be configured to receive user input via one or more buttons, switches, knobs, etc. Additionally, user interface 176 may include a display configured to display information and one or more indicators, such as light indicators, audio indicators, etc., for conveying information and/or providing alerts regarding the operation of pump 100.

Pump 100 may be designed to be insertable into a patient's body, e.g., into a left ventricle of the heart, such as via an introducer system. Although some of the systems and/or methods disclosed herein are described for modulating a pump speed of a pump inserted into the left ventricle of a heart, it should be appreciated that the systems and/or methods described herein may also be applied to other types of ventricular support systems, such as a ventricular support system inserted into the right ventricle of the heart. In one aspect, housing 104, rotor 108, and outflow tube 106 may be radially compressible to enable pump 100 to achieve a relatively small outer diameter of, for example, 9 Fr (3 mm) during insertion. When pump 100 is inserted into the patient, e.g., into a left ventricle, handle 130 and motor 150 may remain disposed outside the patient. As will be appreciated, in other embodiments, the motor of the pump system may be disposed inside the patient upon insertion. During operation, motor 150 is controlled by processor(s) 404 to drive rotation of drive shaft 114 and rotor 108 to convey blood from inlet 116 to outlet 118. It is to be appreciated that rotor 108 may be rotated by motor 150 in reverse to convey blood in the opposite direction (in this case, the openings of 118 form the inlet and the openings of 116 form the outlet). In one aspect, pump 100 may be configured to be used for weeks to months to years to support the heart function of a patient with chronic heart failure, though it should be understood that the technology described herein is not limited to any particular types of procedures and/or use durations.

A blood pump system for use with some embodiments is shown in FIG. 2. In this view, an exemplary intracardiac blood pump assembly 200 adapted for left heart support, in accordance with aspects of the disclosure is depicted. As shown in FIG. 2, an intracardiac blood pump assembly adapted for left heart support may include an elongate catheter 202, a motor 204, a cannula 210, a blood inflow cage 214 arranged at or near the distal end 212 of the cannula 210, a blood outflow cage 206 arranged at or near the proximal end 208 of the cannula 210, and an optional atraumatic extension 216 arranged at the distal end of the blood inflow cage 214. In some embodiments, the blood pump system shown in FIG. 2 may not include an atraumatic extension 216.

In some aspects of the technology, motor 204 may be configured to rotatably drive an impeller (not shown), thereby generating suction sufficient to draw blood into cannula 210 through the blood inflow cage 214, and to expel the blood out of cannula 210 through the blood outflow cage 206. In that regard, the impeller may be positioned distal of the blood outflow cage 206, for example, within the proximal end 208 of the cannula 210 or within a pump housing 207 coupled to the proximal end 208 of the cannula 210. In some aspects of the technology, rather than the impeller being driven by an onboard motor 204, the impeller may instead be coupled to an elongate drive shaft (or drive cable) which is driven by a motor located external to the patient.

Catheter 202 may house electrical lines coupling the motor 204 to one or more electrical controllers and/or sensors. Alternatively, where the impeller is driven by an external motor, an elongate drive shaft may pass through catheter 202. Catheter 202 may also include a purge fluid conduit, a lumen configured to receive a guidewire, etc. In some embodiments, catheter 202 may be coupled to a cable, and the cable may be connected to a control unit. In some embodiments, the control unit described in connection with FIG. 2 may serve the same or similar purpose, and may have the same or similar properties and/or features described with respect to the control unit 170 of the pump system 100 shown in FIG. 1A.

The blood inflow cage 214 may include one or more apertures or openings configured to allow blood to be drawn into cannula 210 when the motor 204 is operating. Likewise, blood outflow cage 206 may include one or more apertures or openings configured to allow blood to flow from the cannula 210 out of the intracardiac blood pump assembly 200. Blood inflow cage 214 and outflow cage 206 may be composed of any suitable bio-compatible material(s). For example, blood inflow cage 214 and/or blood outflow cage 206 may be formed out of bio-compatible metals such as stainless steel, titanium, or biocompatible polymers such as polyurethane. In addition, the surfaces of blood inflow cage 214 and/or blood outflow cage 206 may be treated in various ways, including, but not limited to etching, texturing, or coating or plating with another material. For example, the surfaces of blood inflow cage 214 and/or blood outflow cage 206 may be laser textured.

Cannula 210 may include a flexible hose portion. For example, cannula 210 may be composed, at least in part, of a polyurethane material. In addition, cannula 210 may include a shape-memory material. For example, cannula 210 may comprise a combination of a polyurethane material and one or more strands or coils of a shape-memory material such as Nitinol. Cannula 210 may be formed such that it includes one or more bends or curves in its relaxed state, or it may be configured to be straight in its relaxed state. In that regard, as shown in the exemplary arrangement of FIG. 2, the cannula 210 may have a single pre-formed anatomical bend 218 based on the portion of the left heart in which it is intended to operate. Despite this bend 218, the cannula 210 may nevertheless also be flexible, and may thus be capable of straightening (e.g., during insertion over a guidewire), or bending further (e.g., in a patient whose anatomy has tighter dimensions). Further in that regard, cannula 210 may include a shape-memory material configured to allow the cannula 210 to be a different shape (e.g., straight or mostly straight) at room temperatures, and to form bend 218 once the shape-memory material is exposed to the heat of a patient's body. Cannula 210 may include one or more capacitive sensors 219 associated therewith. As described herein, the capacitive sensor(s) 219 may be configured to sense the presence of one or more objects (e.g., heart wall, heart valve) proximal to cannula 210, and detection of the object(s) may be used to determine the position of the blood pump assembly 200 within a patient.

Atraumatic extension 216 may assist with stabilizing and positioning the intracardiac blood pump assembly 200 in the correct position in the patient's heart. Atraumatic extension 216 may be solid or tubular. If tubular, atraumatic extension 216 may be configured to allow a guidewire to be passed through it to further assist in the positioning of the intracardiac blood pump assembly 200. Atraumatic extension 216 may be any suitable size. For example, atraumatic extension 216 may have an outer diameter in the range of 4-8 Fr. Atraumatic extension 216 may be composed, at least in part, of a flexible material, and may be any suitable shape or configuration such as a straight configuration, a partially curved configuration, a pigtail-shaped configuration as shown in the example of FIG. 2, etc. Atraumatic extension 216 may also have sections with different stiffnesses. For example, atraumatic extension 216 may include a proximal section that is stiff enough to prevent it from buckling, thereby keeping the blood inflow cage 214 in the desired location, and a distal section that is softer and has a lower stiffness, thereby providing an atraumatic tip for contact with a wall of the patient's heart and to allow for guidewire loading. In such a case, the proximal and distal sections of the atraumatic extension 216 may be composed of different materials, or may be composed of the same material with the proximal and distal sections being treated to provide different stiffnesses.

Notwithstanding the foregoing, as mentioned above, atraumatic extension 216 is an optional structure. In that regard, the present technology may also be used with intracardiac blood pump assemblies and other intracardiac devices that include extensions of different types, shapes, materials, and qualities. Likewise, the present technology may be used with intracardiac blood pump assemblies and other intracardiac devices that have no distal extensions of any kind.

As described herein, the intracardiac blood pump assembly 200 may be inserted percutaneously. For example, when used for left heart support, intracardiac blood pump assembly 200 may be inserted via a catheterization procedure through the femoral artery or axillary artery, into the aorta, across the aortic valve, and into the left ventricle. Once positioned in this way, the intracardiac blood pump assembly 200 may deliver blood from the blood inflow cage 214, which sits inside the left ventricle, through cannula 210, to the blood outflow cage 206, which sits inside the ascending aorta. In some aspects of the technology, intracardiac blood pump assembly 200 may be configured such that bend 218 will rest against a predetermined portion of the patient's heart when the intracardiac blood pump assembly 200 is in a desired location. Likewise, the atraumatic extension 216 may be configured such that it rests against a different predetermined portion of the patient's heart when the intracardiac blood pump assembly 200 is in the desired location.

FIG. 3 depicts an exemplary intracardiac blood pump assembly 300 adapted for right heart support, in accordance with aspects of the disclosure. As shown in FIG. 3, an intracardiac blood pump assembly adapted for right heart support may include an elongate catheter 302, a motor 304, a cannula 310, a blood inflow cage 314 arranged at or near the proximal end 308 of the cannula 310, a blood outflow cage 306 arranged at or near the distal end 312 of the cannula 310, and an optional atraumatic extension 316 arranged at the distal end of the blood outflow cage 306.

As with the exemplary assembly of FIG. 2, motor 304 may be configured to rotatably drive an impeller (not shown), thereby generating suction sufficient to draw blood into cannula 310 through the blood inflow cage 314, and to expel the blood out of cannula 310 through the blood outflow cage 306. In that regard, the impeller may be positioned distal of the blood inflow cage 314, for example, within the proximal end 308 of the cannula 310 or within a pump housing 307 coupled to the proximal end 308 of the cannula 310. Here as well, in some aspects of the technology, rather than the impeller being driven by an onboard motor 304, the impeller may instead be coupled to an elongate drive shaft (or drive cable) which is driven by a motor located external to the patient.

The cannula 310 of FIG. 3 may serve the same purpose, and may have the same properties and features described above with respect to cannula 210 of FIG. 2. However, as shown in the exemplary arrangement of FIG. 3, the cannula 310 may have two pre-formed anatomical bends 318 and 320 based on the portion of the right heart in which it is intended to operate. Here again, despite the existence of bends 318 and 320, the cannula 310 may nevertheless also be flexible, and may thus be capable of straightening (e.g., during insertion over a guidewire), or bending further (e.g., in a patient whose anatomy has tighter dimensions). Further in that regard, cannula 310 may include a shape-memory material configured to allow the cannula 310 to be a different shape (e.g., straight or mostly straight) at room temperatures, and to form bends 318 and/or 320 once the shape-memory material is exposed to the heat of a patient's body. Cannula 310 may include one or more capacitive sensors associated therewith. In the example blood pump assembly 300 shown in FIG. 3, cannula 310 is associated with a first capacitive sensor 319 and a second capacitive sensor 321 provided at different locations along cannula 310. As described herein, the capacitive sensors 319, 321 may be configured to sense the presence of one or more objects (e.g., heart wall, heart valve) proximal to cannula 310, and detection of the object(s) may be used to determine the position of the blood pump assembly 300 within a patient.

The catheter 302 and atraumatic extension 316 of FIG. 3 may serve the same purpose and may have the same properties and features described above with respect to catheter 202 and atraumatic extension 216 of FIG. 2. Likewise, other than being located at opposite ends of the cannula from those of FIG. 2, the blood inflow cage 314 and blood outflow cage 306 of FIG. 3 may be similar to the blood inflow cage 214 and blood outflow cage 206 of FIG. 2, and thus may have the same properties and features described above. In some embodiments, catheter 302 may be coupled to a cable, and the cable may be connected to a control unit. In some embodiments, the control unit described in connection with FIG. 3 may serve the same or similar purpose, and may have the same or similar properties and/or features described with respect to the control unit 170 of the pump system 100 shown in FIG. 1A.

Like the exemplary assembly of FIG. 2, the intracardiac blood pump assembly 300 of FIG. 3 may also be inserted percutaneously. For example, when used for right heart support, intracardiac blood pump assembly 300 may be inserted via a catheterization procedure through the femoral vein, into the inferior vena cava, through the right atrium, across the tricuspid valve, into the right ventricle, through the pulmonary valve, and into the pulmonary artery. Once positioned in this way, the intracardiac blood pump assembly 300 may deliver blood from the blood inflow cage 314, which sits inside the inferior vena cava, through cannula 310, to the blood outflow cage 306, which sits inside the pulmonary artery.

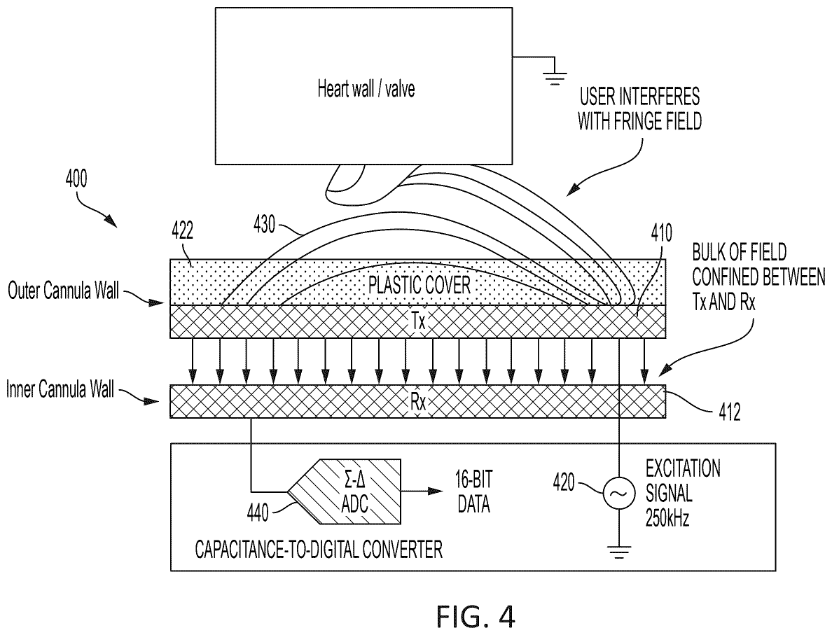

FIG. 4 schematically illustrates a portion of a cannula 400 associated with at least one capacitive sensor, in accordance with some embodiments of the present disclosure. Cannula 400 may include an outer cannula wall 410 and an inner cannula wall 412. A signal generator 420 may be configured to generate an excitation signal having particular frequency characteristics (e.g., a 250 kHz sine wave). The excitation signal may generate an electromagnetic field within the cannula 400. A thin film 422 (e.g., a plastic material) may at least partially surround cannula 400, and may function as a capacitive plate. The electromagnetic field generated by the signal generator 420 may be mostly contained between the outer cannula wall 410 and the inner cannula wall 412 as shown in FIG. 4. However, the signal generator 420 may also generate a fringe field 430 outside of the cannula 400. When an object (e.g., a tissue such as the heart wall, a heart valve, etc.) is present in fringe field 430, the interference in the fringe field 430 caused by the object may alter the capacitance measurement(s) sensed by the capacitance sensor(s). Different objects that enter the fringe field 430 may alter the capacitance measurement(s) in unique ways that are specific to each object. The inventor has recognized and appreciated that the position of the cannula 400 in the patient's anatomy may be determined, based at least in part, on the unique signatures of different objects (e.g., different tissues) being present in the fringe field 430. As shown, a capacitance sensor system designed in accordance with the techniques described herein may measure a capacitance signal and transform the capacitance signal to a digital signal using a capacitance-to-digital converter 440. For example, a capacitance signal may be transformed into 2-bit digital data, 4-bit digital data, 8-bit digital data, 16-bit digital data, etc., and the digital data may be used, at least in part, to determine the position of the pump.

In some embodiments, the capacitance measurement(s) may be determined by measuring changes in the capacitance between the inner cannula wall 412 and the outer cannula wall 410. In such embodiments, the outer cannula wall 410 may be able to move relative to the inner cannula wall 412, resulting in a change in capacitance between the inner cannula wall 412 and the outer cannula wall 410. In such embodiments, when an object comes into contact with the outer cannula wall 410, a capacitance measurement may be determined based on the changed capacitance in the space between the inner cannula wall 412 and the outer cannula wall 410.

In some embodiments, the capacitive sensing system shown in FIG. 4 may include shielding at least partially surrounding one or more of the capacitive sensors to, for example, improve the sensitivity (e.g., signal-to-noise (SNR) ratio) of the capacitive sensor(s). For instance, one or more electrical wires associated with the capacitive sensor(s) may be at least partially surrounded by an electrically insulating sheath to shield the sensor(s). In some embodiments, at least a portion of the catheter may include shielding. In other embodiments, at least a portion of the cable located within the catheter may include shielding.

Although only a single capacitive sensor may be used, it should be appreciated that in some embodiments, multiple capacitive sensors may be arranged at different locations along the cannula 400, and respective capacitive signals from the multiple capacitive sensors may be used to determine the position of the intracardiac blood pump that includes the cannula. For instance, the cannula may include two capacitive sensors. When a first capacitive sensor senses the presence of a heart valve in the fringe field 430 and a second capacitive sensor senses the presence of the heart wall in the fringe field 430, it may be determined that the blood pump is properly positioned across the valve. In another example, the cannula may include three capacitive sensors arranged along the length of the cannula. When the middle capacitive sensor senses the presence of a heart valve in the fringe field 430 and each of the other capacitive sensors senses the presence of the heart wall in the fringe field 430, it may be determined that the blood pump is properly positioned across the valve. Other configurations of capacitive sensors are also contemplated and the particular configuration used does not limit aspects of the technology described herein.

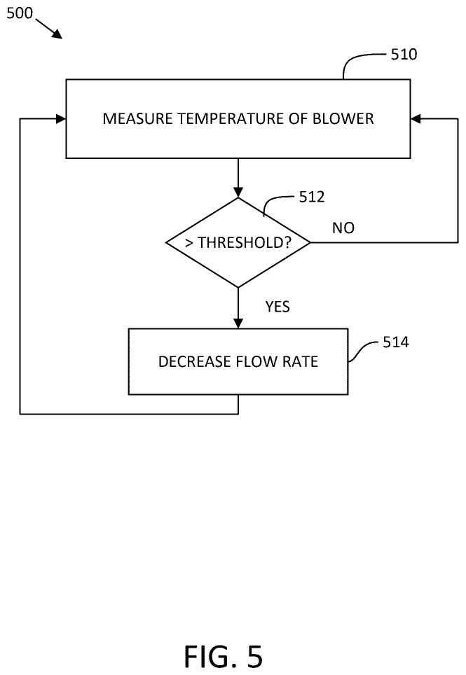

FIG. 5 illustrates a process 500 for determining a position of an intracardiac blood pump in accordance with some embodiments. Process 500 may begin in act 510, where at least one capacitive signal is received from at least one capacitive sensor associated with a cannula of an intracardiac blood pump. For instance, the cannula may include one or more capacitive sensors arranged thereon, an example of which is shown in FIG. 4. In some embodiments, the capacitive signal may reflect the presence of an object (e.g., a tissue) in a fringe electromagnetic field, as described in connection with FIG. 4. In other embodiments, the capacitive signal may reflect contact of the cannula with one or more objects in the patient's vasculature. Contact between the object(s) and the cannula may reduce the distance between the outer and inner walls of the cannula, which may in turn result in a change in the capacitance signal(s) measured by the capacitive sensor(s). The reduction in distance between the outer and inner walls of the cannula may be representative of contact with a particular type of tissue (e.g., a type of tissue having a particular density or other characteristics). As described in connection with FIG. 4, in some embodiments, a single capacitive sensor may be arranged on the cannula, and a corresponding single capacitive signal may be received in act 510. In other embodiments, multiple capacitive sensors may be arranged along the length of the cannula and, accordingly, multiple capacitive signals corresponding to multiple capacitive sensors may be received in act 510.

Process 500 may then proceed to act 520, where a position of the intracardiac blood pump is determined based, at least in part, on the at least one capacitive signal. In some embodiments, a plurality of unique signatures corresponding to different object types (e.g., different tissue types) may be stored, and the position of the intracardiac blood pump may be determined based, at least in part, on the stored signatures and the at least one capacitive signal measured by the capacitive sensor(s). In embodiments in which multiple capacitive signals are received, each of the capacitive signals may be compared with the stored signatures to determine a corresponding tissue type. Additionally or alternatively, at least some the capacitive signals may be combined in some way (e.g., added, subtracted, etc.) and the combined signal may be compared with the stored signatures to determine the position of the blood pump.

In some embodiments, the position of the intracardiac blood pump may be determined based, at least in part, on the capacitive signal(s) without determining a corresponding tissue type located proximal to the capacitive sensor(s). For instance, the system may be configured to store example signatures for good placements of the blood pump (e.g., across the aortic valve) and poor placements of the blood pump (e.g., in the left ventricle), and the capacitive signal(s) may be compared against the stored signatures to determine whether the blood pump is placed in a desired position irrespective of the tissue type impacting the capacitive signal measurements. In some embodiments, the determined position of the intracardiac blood pump may be relative to the patient's anatomy (e.g., the position of the blood pump relative to a heart valve) rather than reflecting an absolute position of the blood pump within the patient's heart. For instance, capacitive coupling of a capacitive sensor with a patient's valve leaflets may be used to determine whether the blood pump is properly positioned across the valve (e.g., across the aortic valve).

Process 500 may then proceed to act 530, where an indication of the position of the intracardiac blood pump may be provided on a user interface associated with the system. For instance, an indication of the pump position may be presented visually on a display of a controller of the blood pump system including the intracardiac blood pump. In some embodiments, the indication of the pump position may be provided audibly, using tactile feedback, or using a combination of auditory, tactile and/or visual feedback. In some embodiments, the techniques described herein may be used to refine or otherwise modify an estimate of pump position determined using other (i.e., non-capacitive sensing) techniques. For example, the pump position may be determined, at least in part, on a motor current signal associated with the motor of the blood pump during operation. Information from the capacitive sensing system described herein may be used as an additional information source to determine the position of the intracardiac blood pump estimated based on the motor current signal.

It should be appreciated that process 500 may be performed at any suitable time and/or at any suitable interval at which determining the position of an intracardiac blood pump is desired. For instance, the position of the blood pump may be determined using one or more of the techniques described herein as a physician is inserting the blood pump into the patient's heart through the patient's vasculature (e.g., prior to operation of the pump). As the physician advances the blood pump through the patient's vasculature, the capacitive signal(s) sensed by the capacitive sensor(s) may change, and the change in capacitive measurement(s) may be used to determine the position of the pump to facilitate proper placement of the pump. In another example, the position of the pump may be determined using one or more of the techniques described herein during operation of the pump. For instance, although the pump may have been initially placed in a desired position (e.g., across the aortic or pulmonary valve), over time the position of the blood pump in the patient's heart may change. The sensing techniques described herein may be used, at least in part, to detect when the position of the blood pump deviates from the desired position. In such instances, an indication of the deviation may be used to alert a physician or other healthcare provider that the pump should be repositioned. Additionally or alternatively, the sensing techniques described herein may be used to facilitate repositioning of the pump without the use of an imaging system, which is typically required for pump repositioning.

In some embodiments, the capacitive sensing techniques described herein may be used as a diagnostic tool to probe characteristics of the patient's cardiac system. For instance, a controller of the intracardiac blood pump system may store one or more unique signatures of healthy heart wall tissue. When a patient's heart wall tissue enters the fringe electromagnetic field of the capacitive sensing system as described herein, a comparison of the detected capacitive signals and the stored unique signature(s) may provide insight into the healthiness (or some other characteristic) of the patient's heart wall tissue. In some embodiments, the capacitive sensing techniques may be used as a diagnostic tool to enable a physician to track one or more characteristics of the patient's heart over time as the patient is provided with cardiac support by the blood pump by comparing capacitive measurements made at different points in time (e.g., during the course of the patient's treatment with the intracardiac blood pump).

Having thus described several aspects and embodiments of the technology set forth in the disclosure, it is to be appreciated that various alterations, modifications, and improvements will readily occur to those skilled in the art. Such alterations, modifications, and improvements are intended to be within the spirit and scope of the technology described herein. For example, those of ordinary skill in the art will readily envision a variety of other means and/or structures for performing the function and/or obtaining the results and/or one or more of the advantages described herein, and each of such variations and/or modifications is deemed to be within the scope of the embodiments described herein. Those skilled in the art will recognize, or be able to ascertain using no more than routine experimentation, many equivalents to the specific embodiments described herein. It is, therefore, to be understood that the foregoing embodiments are presented by way of example only and that inventive embodiments may be practiced otherwise than as specifically described. In addition, any combination of two or more features, systems, articles, materials, kits, and/or methods described herein, if such features, systems, articles, materials, kits, and/or methods are not mutually inconsistent, is included within the scope of the present disclosure.

The above-described embodiments can be implemented in any of numerous ways. One or more aspects and embodiments of the present disclosure involving the performance of processes or methods may utilize program instructions executable by a device (e.g., a computer, a processor, or other device) to perform, or control performance of, the processes or methods. In this respect, various inventive concepts may be embodied as a computer readable storage medium (or multiple computer readable storage media) (e.g., a computer memory, one or more floppy discs, compact discs, optical discs, magnetic tapes, flash memories, circuit configurations in Field Programmable Gate Arrays or other semiconductor devices, or other tangible computer storage medium) encoded with one or more programs that, when executed on one or more computers or other processors, perform methods that implement one or more of the various embodiments described above. The computer readable medium or media can be transportable, such that the program or programs stored thereon can be loaded onto one or more different computers or other processors to implement various ones of the aspects described above. In some embodiments, computer readable media may be non-transitory media.

The above-described embodiments of the present technology can be implemented in any of numerous ways. For example, the embodiments may be implemented using hardware, software or a combination thereof. When implemented in software, the software code can be executed on any suitable processor or collection of processors, whether provided in a single computer or distributed among multiple computers. It should be appreciated that any component or collection of components that perform the functions described above can be generically considered as a controller that controls the above-described function. A controller can be implemented in numerous ways, such as with dedicated hardware, or with general purpose hardware (e.g., one or more processor) that is programmed using microcode or software to perform the functions recited above, and may be implemented in a combination of ways when the controller corresponds to multiple components of a system.

Further, it should be appreciated that a computer may be embodied in any of a number of forms, such as a rack-mounted computer, a desktop computer, a laptop computer, or a tablet computer, as non-limiting examples. Additionally, a computer may be embedded in a device not generally regarded as a computer but with suitable processing capabilities, including a Personal Digital Assistant (PDA), a smartphone or any other suitable portable or fixed electronic device. Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible formats.

Such computers may be interconnected by one or more networks in any suitable form, including a local area network or a wide area network, such as an enterprise network, and intelligent network (IN) or the Internet. Such networks may be based on any suitable technology and may operate according to any suitable protocol and may include wireless networks, wired networks or fiber optic networks.

Also, as described, some aspects may be embodied as one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification, unless clearly indicated to the contrary, should be understood to mean “at least one.”

The phrase “and/or,” as used herein in the specification should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

In the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

1. A circulatory support device, comprising:
an intracardiac blood pump comprising an inlet, an outlet, and a cannula arranged between the inlet and the outlet;
at least one capacitive sensor associated with the cannula; and
at least one controller configured to:
determine a position of the intracardiac blood pump in a patient based, at least in part, on at least one capacitive signal sensed by the at least one capacitive sensor; and
provide an indication of the position of the intracardiac blood pump on a user interface of the circulatory support device.

2. The circulatory support device of claim 1, further comprising:
a capacitive material surrounding the cannula; and
a signal generator configured to impart an electromagnetic field surrounding the capacitive material.

3. The circulatory support device of claim 2, wherein the at least one capacitive signal corresponds to at least one interference signal sensed when an object enters the electromagnetic field.

4. The circulatory support device of claim 3, further comprising an analog-to-digital converter configured to transform the at least one interference signal to a digital signal,
wherein the at least one controller is further configured to determine the position of the intracardiac blood pump based, at least in part, on the digital signal.

5. The circulatory support device of claim 1, wherein the at least one controller is further configured to determine the position of the intracardiac blood pump based, at least in part, on a comparison of the at least one capacitive signal with plurality of stored signal representations.

6. The circulatory support device of claim 1, further comprising:
a shielding material arranged adjacent to the at least one capacitive sensor.

7. The circulatory support device of claim 1, wherein
the at least one capacitive sensor comprises a plurality of capacitive sensors, each of which is configured to sense a respective capacitive signal, and
the at least one controller is further configured to determine a position of the intracardiac blood pump in a patient based, at least in part, on the plurality of capacitive signals.

8. The circulatory support device of claim 7, wherein the at least one controller is further configured to determine a position of the intracardiac blood pump in a patient based, at least in part, on the plurality of capacitive signals by:
comparing each of the plurality of capacitive signals to a stored signal representation; and
determining the position of the intracardiac blood pump based, at least in part, on the comparisons.

9. The circulatory support device of claim 7, wherein the at least one controller is further configured to determine a position of the intracardiac blood pump in a patient based, at least in part, on the plurality of capacitive signals by:
combining each of the plurality of capacitive signals to generate a combined signal; and
determining the position of the intracardiac blood pump based, at least in part, on a comparison of the combined signal with plurality of stored signal representations.

10. The circulatory support device of claim 1, wherein the at least one controller is further configured to:
determine based, at least in part, on the at least one capacitive signal, an anatomy type of a tissue proximal to the cannula.

11. The circulatory support device of claim 10, wherein the at least one controller is further configured to:
determine a position of the intracardiac blood pump in a patient based, at least in part, on the determined anatomy type.

12. A method of determining a position of an intracardiac blood pump in a patient, the method comprising:
receiving from at least one capacitive sensor associated with a cannula of the intracardiac blood pump, at least one capacitive signal;
determining a position of the intracardiac blood pump in the patient based, at least in part, on the at least one capacitive signal; and
providing an indication of the position of the intracardiac blood pump on a user interface.

13. The method of claim 12, further comprising:
generating, using a signal generator, an electromagnetic field proximate to the cannula,
wherein the at least one capacitive signal corresponds to at least one interference signal sensed when an object enters the electromagnetic field.

14. The method of claim 13, further comprising
transforming the at least one interference signal to a digital signal,
wherein determining the position of the intracardiac blood pump comprises determining the position of the intracardiac blood pump based, at least in part, on the digital signal.

15. The method of claim 12, wherein determining the position of the intracardiac blood pump comprises determining the position of the intracardiac blood pump based, at least in part, on a comparison of the at least one capacitive signal with plurality of stored signal representations.

16. The method of claim 12, wherein
receiving at least one capacitive signal comprises receiving a plurality of capacitive signals from a plurality of capacitive sensors associated with the cannula of the intracardiac blood pump, and
determining a position of the intracardiac blood pump comprises determining the position in a patient based, at least in part, on the plurality of capacitive signals.

17. The method of claim 16, wherein determining a position of the intracardiac blood pump comprises determining the position of the intracardiac blood pump by:
comparing each of the plurality of capacitive signals to a stored signal representation; and
determining the position of the intracardiac blood pump based, at least in part, on the comparisons.

18. The method of claim 16, wherein determining a position of the intracardiac blood pump comprises determining the position of the intracardiac blood pump by:
combining each of the plurality of capacitive signals to generate a combined signal; and
determining the position of the intracardiac blood pump based, at least in part, on a comparison of the combined signal with plurality of stored signal representations.

19. The method of claim 12, further comprising:
determining based, at least in part, on the at least one capacitive signal, an anatomy type of a tissue proximal to the cannula.

20. The method of claim 19, wherein determining a position of the intracardiac blood pump comprises determining the position of the intracardiac blood pump based, at least in part, on the determined anatomy type.
